(19) United States
(12) Patent Application Publication
(10) Pub. No.: US 2015/0173910 A1
(43) Pub. Date: Jun. 25, 2015

(54) METHOD AND SYSTEM FOR IMPLANTS

(71) Applicant: NLT SPINE LTD, KFAR SABA (IL)

(72) Inventors: Tzony Siegal, Moshav Shoeva (IL); Oded Loebl, Tel Mond (IL); Didier Toubia, Raanana (IL)

(21) Appl. No.: 14/421,844

(22) PCT Filed: Aug. 29, 2013
(86) PCT No.: PCT/IB2013/058104

§ 371 Date: Feb. 16, 2015

Related U.S. Application Data
(60) Provisional application No. 61/694,279, filed on Aug. 29, 2012.

(51) Int. Cl. A61F 2/44

(52) U.S. Cl. CPC A61F 2/442

(57) ABSTRACT

A surgical implant includes a rod or guide, initially deployed to the surgical site, over which segments are deployed, typically by sliding and advancing the segments along or over the rod or guide. The rod is typically of a predefined curvature, which defines the shape of the implant. The rod deflects or orients into this predefined curvature upon its deployment at the surgical site. A portion of the rod with the segments remains at the surgical site after the requisite procedure is performed.

### CROSS-REFERENCES TO RELATED APPLICATIONS

This application is related to and claims priority from commonly owned U.S. Provisional Patent Application Ser. No. 61/694,279, entitled: Segments on Rail, filed Aug. 29, 2012, the disclosure of which is incorporated by reference in its entirety herein.

### TECHNICAL FIELD

The present invention is directed implants and devices for deploying implants in the body, such as, between vertebrae.

### BACKGROUND

Spinal procedures to stabilize vertebrae, including procedures such as spinal fusions, require surgeons introducing devices into the body. The devices include intervertebral implants, which maintain the relative position of the vertebrae during the healing phase of the fusion, as well as serving as a boundary for bone cement or other compositions used for facilitating the fusion between two vertebrae.

### SUMMARY

The present invention is directed to a device for deployment into the body, such as an implant, which, for example, uses a flexible rod, or guide, for example, in the shape of a rail. The rail is deployed, for example, into the intervertebral disc space, in a straight or otherwise aligned or axial configuration, and once in the body, takes on a curved shape. Segments are then advanced over the rail. Once the requisite number of segments have been advanced, and the desired implant (apparatus) is created, additional procedures may be performed using the implant, such as filling the area with bone fill or other fusion promoting material.

The present invention is directed to an implant (apparatus) formed of a rod or guide, initially deployed to the surgical site, over which segments are deployed, typically by sliding and advancing along or over the rod or guide. The rod is typically of a predefined curvature, which defines the shape of the implant. The rod deflects or orients into this predefined curvature upon its deployment at the surgical site. The rod, i.e., a portion of the rod, with the segments remains at the surgical site after the requisite procedure is performed.

A disclosed embodiment is directed to a surgical implant for use in the body. The implant comprises: a rod or guide including a first section configured for deflecting into a predetermined non-linear shape when deployed in the body, from a non-deflected substantially linear shape, and a second section, the rod configured to be separated between the first section and the second section; and, at least one segment configured for movement, for example, sliding, along the first and second sections of the rod, and configured to conform with the non-linear shape, e.g., curved coiled or rounded, of the first section of the rod for positioning thereon, when the first section of the rod is deflected upon being deployed in the body. The second section of the rod is, for example, linear, and, for example, rigid. The rod is such that an engagement portion, for example, a rail, such as in a “T” shape, or a groove, for example, of a “U” shaped cross section, extends along the rod, and the at least one segment is correspondingly configured to the cross sectional shape of the engagement portion for slideably interlocking with the engagement portion along the rod. For example, the engagement portion of the rod is on the exterior surface of the rod. Also, for example, the engagement portion of the rod is on the interior surface of the rod. The first section of the rod includes, for example, a shape-memory material which is configured to deflect into the predetermined non-linear shape when deployed in the body.

The surgical implant, which is designed to remain in the body, is formed, for example, by the first (curved, coiled or rounded) section of the rod, and the at least one segment thereon, but typically, a plurality of segments are positioned along the first section rod.

For example, the first section of the rod may include a plurality of flexibly connected links. These links, for example, may be of the same length. Also, for example, the at least one segment is of a length greater than the combined length of the links. Still also, for example, the at least one segment includes a plurality of segments, each of the segments of a length such that each of the separations between the links are overlapped by a segment of the plurality of segments.

The at least one segment, and the plurality of segments include slots, which, for example, may be of a shape correspondingly configured to the “T” shape of the engagement portion of the rod. For example, the slot of each segment extends in an arc therethrough. Also, for example, the slot can extend linearly through each segment. Additionally, for example, when the surgical implant is formed from a plurality of segments, each of the segments may include correspondingly configured engaging structures at oppositely disposed ends of the segment.

The rod or guide may also be an I-beam shaped member, which accommodates a plurality of segments, each of the segments including slots at oppositely disposed ends to engage a portion of the I-beam shaped member. The slots, for example, extend linearly through each segment. Also, for example, the slots may extend in an arc through each segment.

Another embodiment disclosed is directed to a method for deploying a surgical implant to a surgical site in the body. The method comprises: accessing the surgical site with a tube or other conduit; moving a rod or guide through the tube, whereby the leading section of the rod deflects into a predetermined non-linear shape, e.g., curved, coiled or rounded, upon exiting the tube at the surgical site; removing the tube from enveloping the rod; moving, e.g., sliding or placing, at least one segment along the rod toward the edge of the leading end, to define the surgical implant; separating a section of the rod from the leading section of the rod; and, removing the separated section of the rod from the body. The at least one segment includes a plurality of segments, and moving the at least one segment includes moving, for example, by sliding the segment(s) along the rod distally, each segment of the plurality of segments in succession until the leading (distal-most) segment is at the edge of the leading or distal end of the rod, with the segments in abutment with each other. Each segment of the plurality of segments may be configured to engage a successive segment of the plurality of segments in an interlocking manner at the oppositely disposed ends of the segments, such that the segments are interlocked, and their movement is inhibited.

### BRIEF DESCRIPTION OF THE DRAWINGS

Attention is now directed to the drawings, where like reference numerals or characters indicate corresponding or like components. In the drawings:

FIGS. 1A and 1B are perspective views of a rod in the form of a rail in an uncoiled or straightened position, in accordance with an embodiment of the invention;

FIG. 2 is a perspective view of the rod of FIGS. 1A and 1B in a curved position;

FIGS. 3 and 4 are perspective views of alternative rods with rails in accordance with embodiments of the present invention;

FIGS. 5A and 5B are perspective views of segments used with the rods of FIGS. 1A, 1B and 2-4;

FIG. 6 is a perspective view of an alternative segment for use with the rails of FIGS. 1A, 1B, and 2-4;

FIGS. 7A-7C are cross sections of alternative rods and segments in accordance with other embodiments of the present invention;

FIGS. 8A-8D are perspective views showing deployment of segments on a rod, such as that shown in FIGS. 1A, 1B, and 2-4, at various stages, in accordance with the present invention;

FIGS. 9A and 9B are perspective views showing an alternative rail with segments in accordance with an embodiment of the invention;

FIG. 10 is a perspective view of a series of interlocking rods in the form of U-beams, in accordance with an embodiment of the present invention;

FIG. 11 is a side view detailing the interlocking U-beams of FIG. 10;

FIGS. 12A and 12B are perspective views of I-beam rails with segments, in accordance with an embodiment of the invention;

FIGS. 13A and 13C are perspective views of external facing T-beam rails with segments, in accordance with an embodiment of the invention;

FIG. 13B is a top view of FIGS. 13A and 13C;

FIG. 13D is a cross sectional view along line 13D-13D of FIG. 13C;

FIGS. 14A and 14B are front and rear perspective views of the segments of FIGS. 13A-13D;

FIG. 14C shows an alternative segment in accordance with the segments of FIGS. 14A and 14B;

FIG. 15 is a perspective view of alternative internal facing “T” shaped rail in accordance with an embodiment of the invention;

FIGS. 16A-16C are perspective views of an alternative rod and segments in accordance with an embodiment of the invention;

FIGS. 17A-17G are diagrams detailing deployment of an apparatus in accordance with the present invention;

FIGS. 18-20 are diagrams detailing other deployments of the apparatus in accordance with FIGS. 17A-170; and

FIGS. 21A-21C are diagrams detailing another deployment of an apparatus in accordance with the present invention;

### DETAILED DESCRIPTION OF THE DRAWINGS

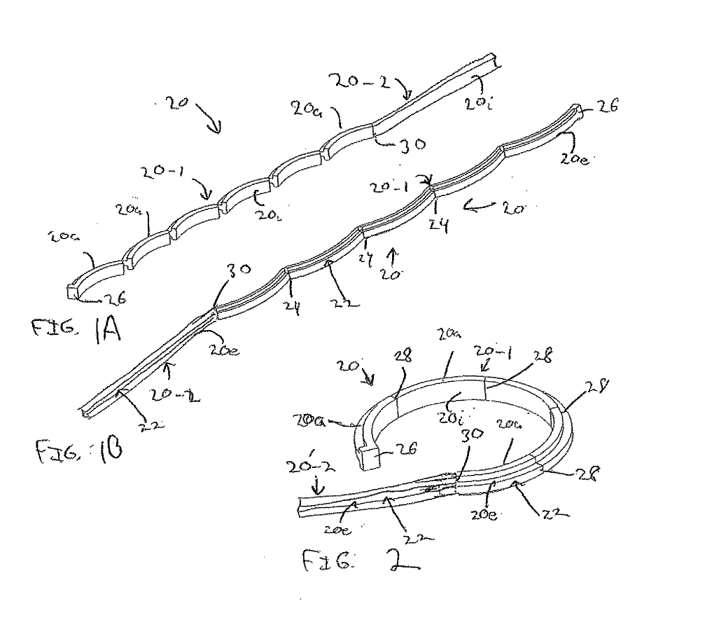

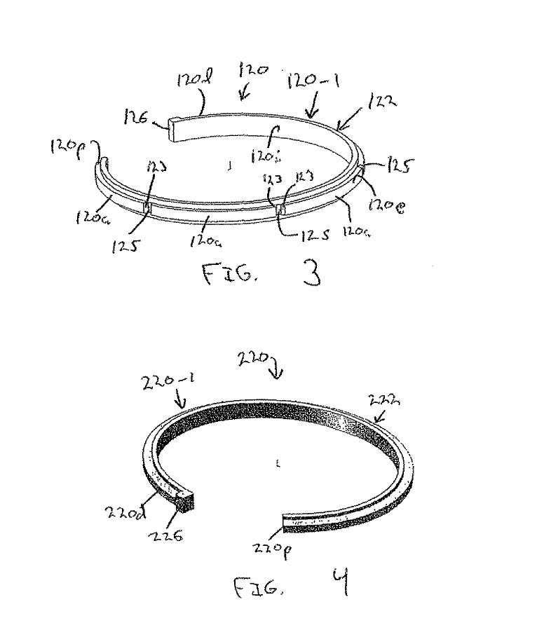

FIGS. 1A and 1B show a rod or guide 20 (hereinafter, collectively referred to as a “rod”) in an extended or uncoiled position, such as that used during deployment (detailed below). FIG. 2 shows the rod 20 in a curved, coiled or rounded position, such as when it is deployed in the body.

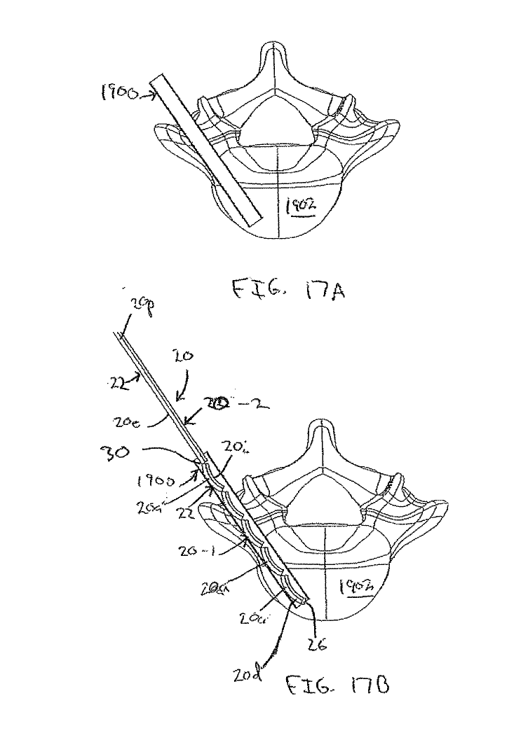

The rod 20 is, for example, an integral member, formed of two sections 20-1 and 20-2. A first, curved, coiled, or rounded (collectively referred to as “curved” hereinafter) section 20-1 is joined to a second or straight section 20-2. The straight section 20-2 is, for example, rigid. The rod 20 is, for example, in the form of a rail 22, such as a “T” shaped (T-shaped) rail with the “T” extending outward or externally. The rail 22 serves as an engaging portion for the rod 20, for segments 50, as detailed below. The rail 22 extends along the external or exterior surface 20e of the rod 20 curved 20-1 and straight 20-2 sections. The rail 22 is also positionable on the inner surface 20i, for example, as shown in FIG. 15, and detailed below.

The curved section 20-1 is, for example, initially linear, substantially linear, or otherwise non-deflected, before deflecting into the curved shape, as shown in FIG. 2, as upon deployment into the body. This coiled, rounded or curved shape is typically preformed or predetermined in the material used to make the rod 20. Additional suitable curved or coiled shapes for the curved section 20-1 include, for example, circular, oval, and the like.

The curved section 20-2 is, for example, formed of links 20a. Each link 20a is attached to a successive link 20a at a junction or joint 24 along the exterior surface 20e, where the links 20a are connected in a flexible manner. For example, the links 20a are connected in a hinged manner, as shown in FIG. 1B, by the resilience of the joints 24. The joints 24 exhibit spring-like or flexible behavior, such that links 20a move between the uncoiled (FIGS. 1A and 1B) and coiled or curved positions, one such coiled or curved position shown in FIG. 2.

A first or proximal end 20p of the rod 20 is open and of a “T” shaped cross section, to receive segments (detailed below) along the rail 22, while the second, distal or opposite end 20d includes a portion, which is rectangular, square, or other shape in cross section, and serves as a stop surface 26 or limiter for segments which are moved along the rod 20 (rail 22), as detailed below. In this curved position, the links 20a abut each other at the joints 24, and form a flush or smooth inner surface 20i of the rod 20, with separations 28 (representative of the joints 24) between the links 20a on the interior surface 20i of the rod 20.

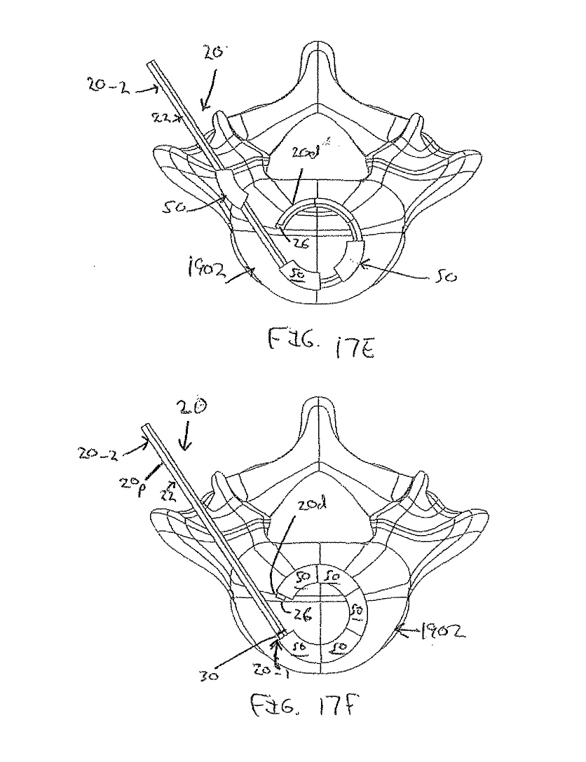

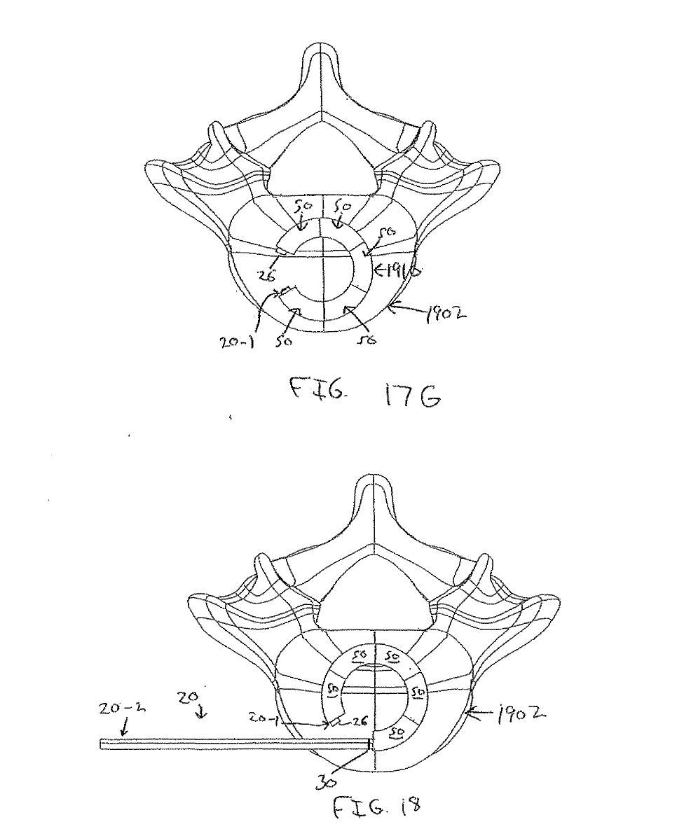

The rod 20 is configured to be separated between the sections 20-1, 20-2, at a weakened portion 30. The straight section 20-2 is designed to be detached (separated) from the coiled section 20-1 once the rod 20 with segments 50 (detailed below) is deployed in the body, as detailed below. The weakened portion 30 of the rod 20 allows for manual separation of the straight section 20-2 from the curved section 20-1, typically upon deployment in the body, by breaking off the straight section 20-2 from the curved section 20-1, and as detailed below. The weakened portion 30 is such that upon the breaking away, one or more edges of the curved section 20-1 extend outward, serving as a proximal stop for the segments, e.g., segments 50 (as detailed below) as shown in FIG. 17G and detailed below. Alternately, the final segment, similar to segments 50, 50′ detailed below, which serve as the proximal most segment, may have a locking mechanism, for example, a spring-loaded tooth, ball, and notch, pin and hole or other, with the first section 20-1, such that motion between the first section 20-1 and the proximal-most segment is limited.

Additionally, the straight section 20-2 is separable and detachable from the curved section 20-1 by arrangements such as: 1) a friction fit between a male (pin) and a female (hole) on the straight 20-2 and curved 20-1 sections, which are separable; 2) the straight 20-2 and curved 20-1 sections are joined by spot welds, which are severable by a predetermined or “breakaway” force; and, 3) any other mechanical, magnetic, geometric, pneumatic, hydraulic locking/holding mechanism, which are separable.

The rod 20 is made of materials, such as shape retaining and/or super elastic materials including NITINOL (nickel-titanium alloy). Other suitable materials include resilient materials such as surgical grade plastics and metals, and the like.

Attention is now directed to FIGS. 3 and 4 which detail alternative curved sections 120-1, 220-1 of rods 120, 220, which are used with the straight section 20-2 of the rod 20, in accordance with the disclosure of rod 20 above and as shown in FIGS. 1A, 1B and 2.

FIG. 3 shows an alternative rod 120, for example, the curved section 120-1, formed as a rail 122 of an external “T” shape. The rod 120 includes links 120a, which are formed by outwardly tapered cuts 123, with gaps 125 defining spaces between the links 120a. These gaps 125 provide a space for flexing the links 120a outward into a straightened configuration, similar to that shown in FIGS. 1A and IB. The rod 120 is made of the materials disclosed above for the rod 20. Except where indicated, the rod 120 is similar in construction and function to the rod 20, with similar elements increased by “100”.

FIG. 4 shows an alternative rod 220, for example, the curved section 220-1, formed as a rail 222 of an external “T” shape. The rod 220 is made of the materials disclosed above for the rod 20. Except where indicated, the rod 220 is similar in construction and function to the rod 20, with similar elements increased by “200”. The rod 220 is continuous. A first or proximal end 220p of the rod 220 is open and of a “T” shaped cross section, to receive segments (detailed below) along the rail 222, while the second, distal or opposite end 220d includes a terminal portion which is rectangular or square in cross section, to serve as a stop surface 226 or limiter for segments which are moved along the rod 220 (rail 222), as detailed below.

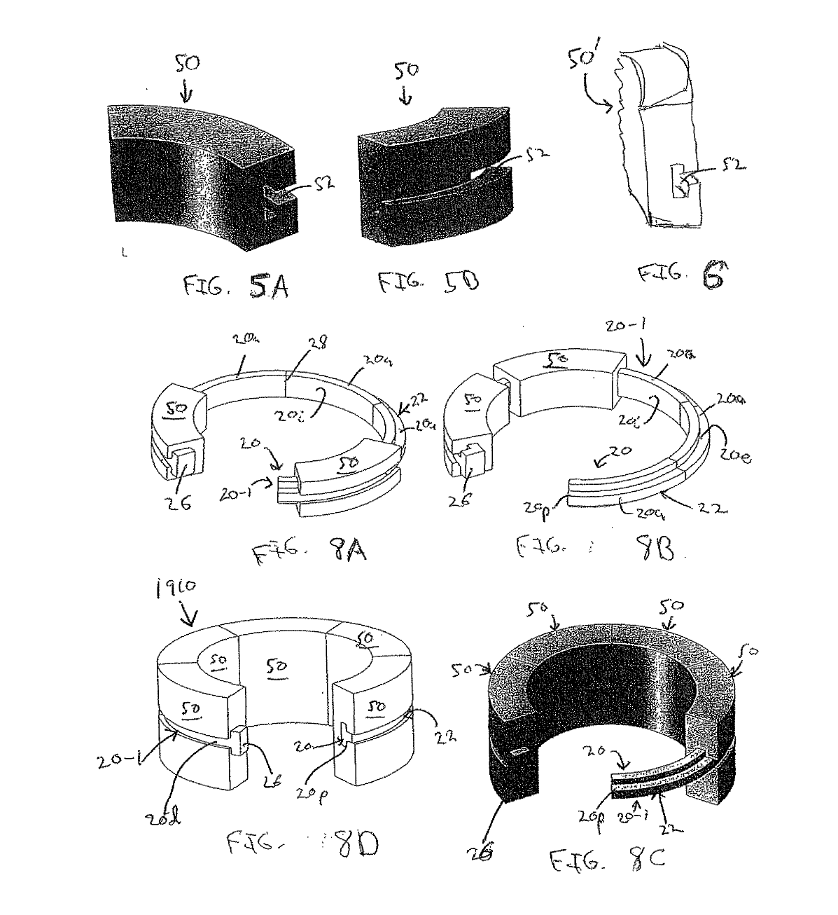

FIGS. 5A and 5B show segments 50, for example, for use with the rods 20, 120, 220, as well as other rods with an external “T” shaped exterior surface. The segments 50 include an interior slot or groove 52 of a shape corresponding to that of the rail 22, which is a “T” shape. For example, the interior slot 52 may be curved, as shown in FIGS. 5A and 5B, with the slot 52 extending through the segment 50 in an arc, the arc of a curvature slightly greater than the curvature of the rail 22 (to maintain the slideable interlocking between the rod 20 and the respective segments 50). Alternatively, the “T” shape of the slot 52 may be linear, as the slot 52 extends linearly through the segment 50). This construction for the slots 52 of the segments 50 is also applicable to and usable with rods 20, 120, 220 and rails 22, 122, 222. The interior slot 52 is dimensioned to allow each segment 50 to slide along the respective rails 22, 122, 222, with a frictionally tight, yet slideable, tolerance. The interior slot 52 is centrally located in the segment 50.

FIG. 6 shows an alternative segment 50′ which is similar to the segments 50 detailed above. The segment 50′ differs from segments 50 detailed above in that the interior slot 52 of the segment 50′ is located off-center, for example, below the center line, as shown, but may be above the center line. These alternative segments 50′, for example, are for use with the rods 20, 120, 220, as well as other rods with an external “T” shaped exterior surface.

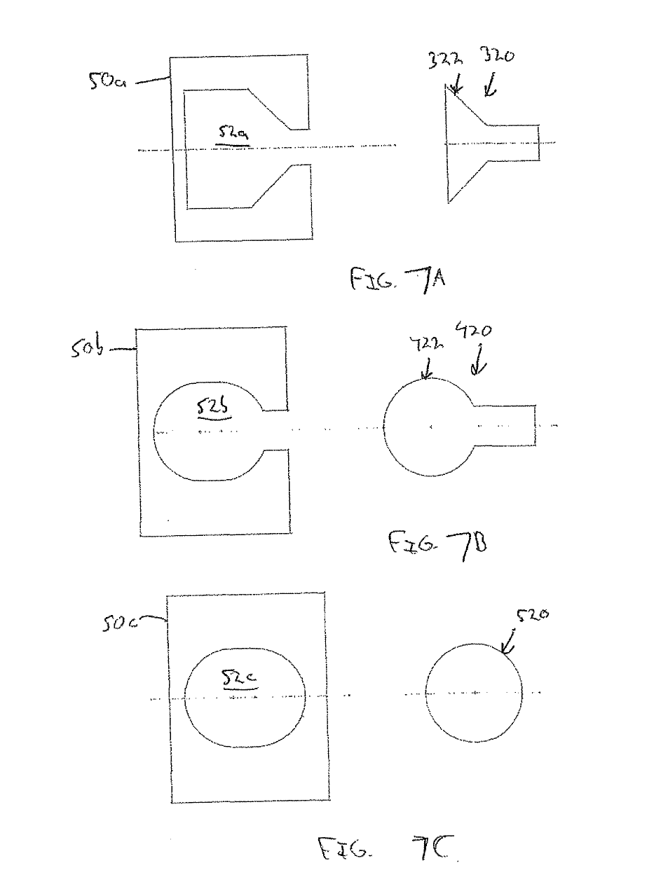

Also alternatively, for example, as shown in FIGS. 7A-7C, segments 50a, 50b and 50c may be designed in accordance with the segments 50, 50′, but with “Y” shaped slots 52a (FIG. 7A) to accommodate a rod 320 with a “Y” shaped rail 322, a rounded shape slot 52b (FIG. 7B) to accommodate a rod 420 with a rounded-end rail 422, and, an internal enclosed rounded slot 52c (FIG. 7C) to accommodate a rod 520 which is rounded. Rods 320, 420 and 520 are similar in construction to rod 20 described above.

While multiple segments 50, 50′, 50a, 50b, 50c are described, a single segment of the desired length/arc length is also permissible. Alternatively, a plurality of segments 50, 50′ may joined, with flexible connections between the segments, so as to be part of an integral member, slid over or placed on a rod, so as to form the implant. The segments that make up the implant would be on one or more integral members and could be combined with individual segments as desired, depending on the desired implant. Alternatively, while the aforementioned segments are designed for being advanced over the respective rods by sliding, the can also be friction fitted, or “snapped” onto the rod, at the desired position (except for rod 520).

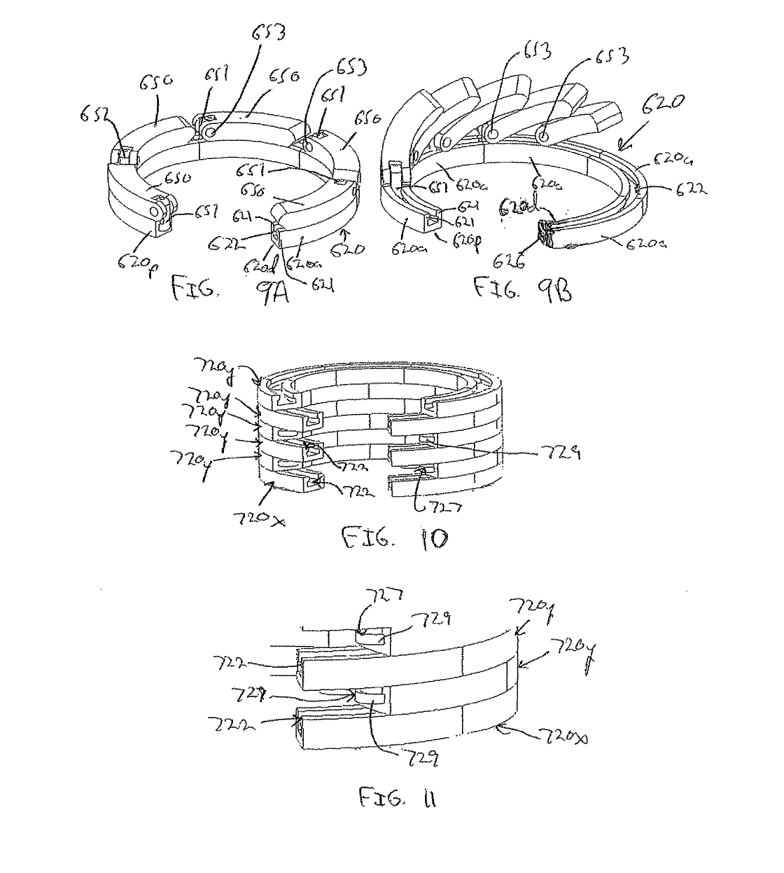

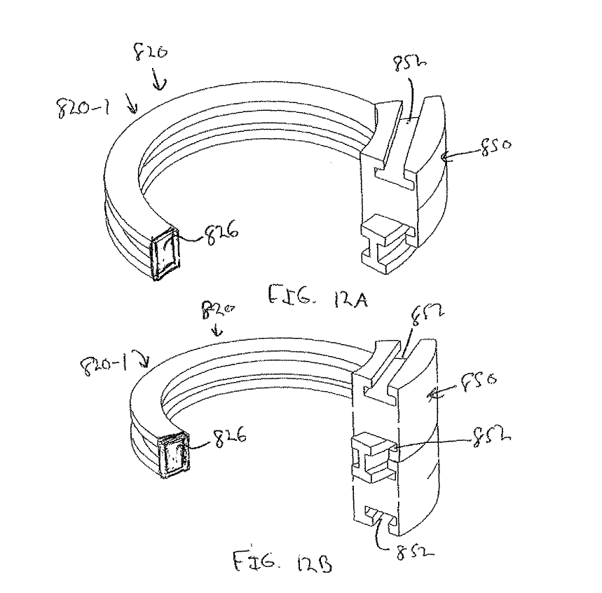

FIGS. 8A-8D illustrate segments 50 being deployed on the rod 20 in a progression, or alternatively, the curved section 20-1 of the rod 20, the rod 20 having a “T” shaped rail 22. The segments 50, upon being received at the proximal end 20p of the rod 20 on the rail 22, are advanced (toward the distal end 20d of the rod 20) by being slid over the rod 20 distally, as the slots 52 of the segments 50 slideably engage the rail 22, and accordingly, slideably engage at least a portion of the rod 20. The slot 52 of the segment 50 receives the correspondingly shaped rail 22. Segments 50 are advanced distally, toward the distal end 20d of the rail 22. The distal-most segment 50 is advanced until it reaches the stop surface 26 of the distal end 20d of the rail 22, typically abutting the stop surface 26, such that further distal movement of the segment 50 is no longer possible, as it is blocked by the stop surface 26.

Successive segments 50 are advanced distally, in a progressive manner, typically until segments 50 abut each other, such that their movement is substantially limited, as shown in FIG. 8D. As shown in FIG. 8D, an implant or apparatus 1910 is now complete. The segments 50 are, for example, of a different length, either greater or less than the length of each link 20a. Accordingly, the segments 50 are of sufficient length, such that a segment overlaps each of the separations 28 between the links 20a. This overlap locks the rod 20, at the curved section 20-1, in its curved, coiled or rounded position, maintaining the curved, coiled, or otherwise non-linear shape (to which the curved section 20-1 has deflected) of the curved section 20-1 of the rod 20. This avoids any potential uncoiling of the rod 20, when the implant (apparatus) 1910 (rod 20 and segments 50) are deployed in the body, for example, as detailed below.

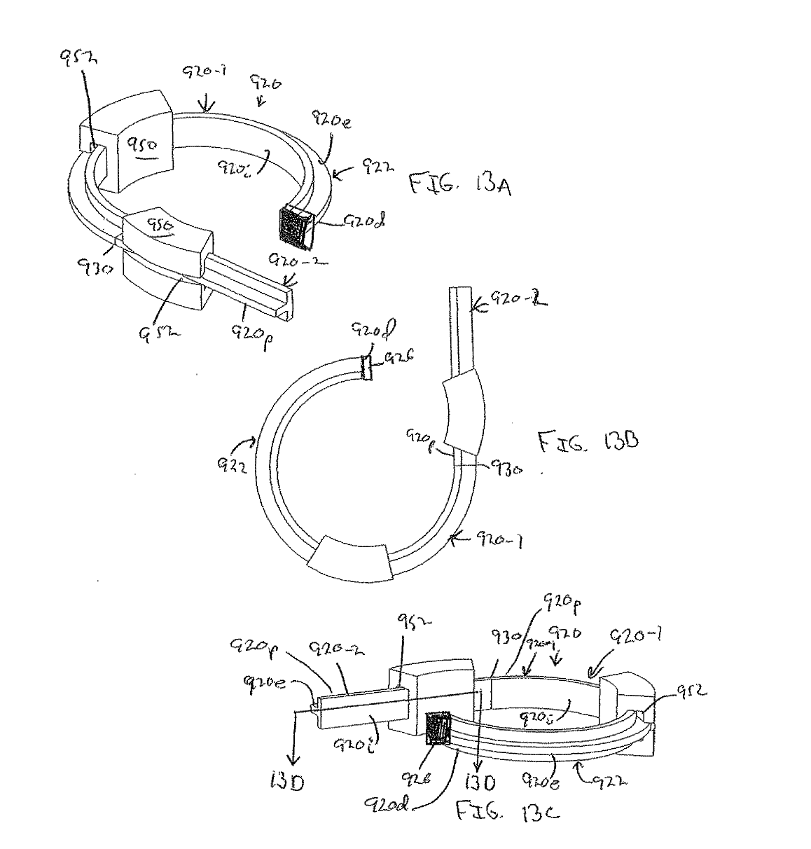

FIGS. 9A and 9B show an alternative embodiment of a rod 620, for example, the curved section 620-1 thereof, with a track 622, which has a U-shaped cross section (hereinafter, a U-shaped track) extending into the rod 620. The rod 620 is in links 620a similar to rod 20 with its links 20a. The rod 620 typically also includes a straight section, like that of the rod 20, but with the U-shaped track.

The “U” shape includes inwardly directed shoulders 621 to retain the correspondingly shaped (for example, “T” shaped) protruding members 651 of each of the stacking segments 650, which move within the track 622. Although not shown, the track 622 includes a stop surface 626 (FIG. 9B, similar to stop surface 26), at its distal end 620d. Except where indicated, the rod 620 is similar in construction and function to the rod 20, with similar elements increased by “600”.

The stacking segments 650 are hinged at one end, although hinging at both ends is also permissible, to a protruding member 651 by a pivotal hinge 653. The stacking segments 650 can be stacked one over another, as shown in FIG. 9B, and when moved distally, or the segments 650 otherwise spread apart from each other, proximally and/or distally, cover the rod 620, so as to be properly deployed for surgical use, as shown in FIG. 9A. One or more of the segments 650 can be connected to a string, wire, or other pulling/pushing mechanism to spread the segments 650 (FIG. 9A) or stack the segments 650 (FIG. 9B).

FIGS. 10 and 11 show stacked and interlocking rods, for example, in the form of “U” shaped beams 720x and 720y. The lowermost “U” shaped beam 720x (“lowermost” being an exemplary orientation for description purposes) is identical or similar to the rods 620 with a “U” shaped track 622, detailed above and shown in FIGS. 10 and 11. The remaining “U” shaped beams 720y are similar to the rods 620 with “U” shaped tracks 622, detailed above, but include feet 727 with a lower protrusion 729, shown in detail in FIG. 11, to allow for slideable movement and retention of a successive beam 720y in the respective “U” shaped track 722 of the respective “U” shaped beam 720x, 720y. The “U” shaped beams 720x, 720y are stackable to any height desired, by sliding one beam over another in the distal direction along the respective “U” shaped beam 720x, 720y.

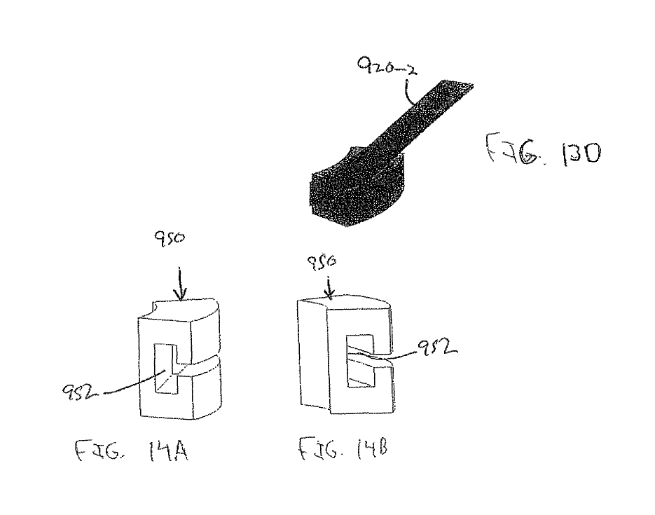

FIGS. 12A and 12B show a rod 820 in the shape of an 1-beam, for example, the curved section 820-1 thereof. The rod 820 typically also includes a straight section, like that of the rod 20, but also in the shape of the I-beam. The rod 820 is of similar construction to the rod 20, with similar components increased by “800”. The I-beam rod 820, for example, accommodates two segments 850, each with two slots 852, on opposite ends of the segments, the slots 852 which are, for example linear. This arrangement of slots 852 allows for multiple stacking of I-beam rods 820 and segments 850. The segments 850 are deployed on the rod 820 in a manner similar to that for segments 50, as shown in FIGS. 8A-8D and described above. The stop surface 826 at the distal end of the rod 820 (820-1), prevents the segments 850 from leaving the rod 820.

Alternatively, segments with I-shaped slots, portions thereof, or are otherwise configured to accommodate the I-beam rods 820 are also permissible.

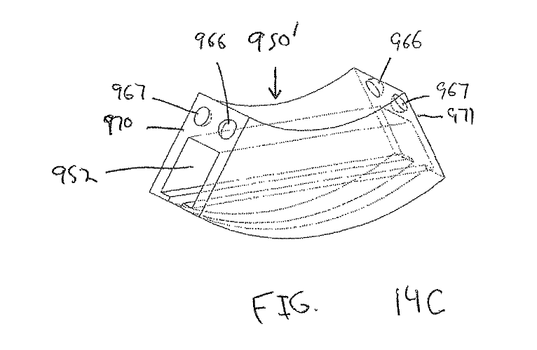

FIGS. 13A-13D show a rod 920 as a “T” shaped rail 922, with a rounded, curved or coiled section 920-1 (terminating in a stop surface 926), and a straight section 920-2. The rod 920 is of similar construction to the rod 20, with similar components increased by “900”. The straight section 920-1 is designed to be broken away from the coiled, curved, or rounded (collectively “curved”) section 920-1, at the weakened portion 930, identical or similar to that of weakened portion 30 of the rod 20, as detailed above.

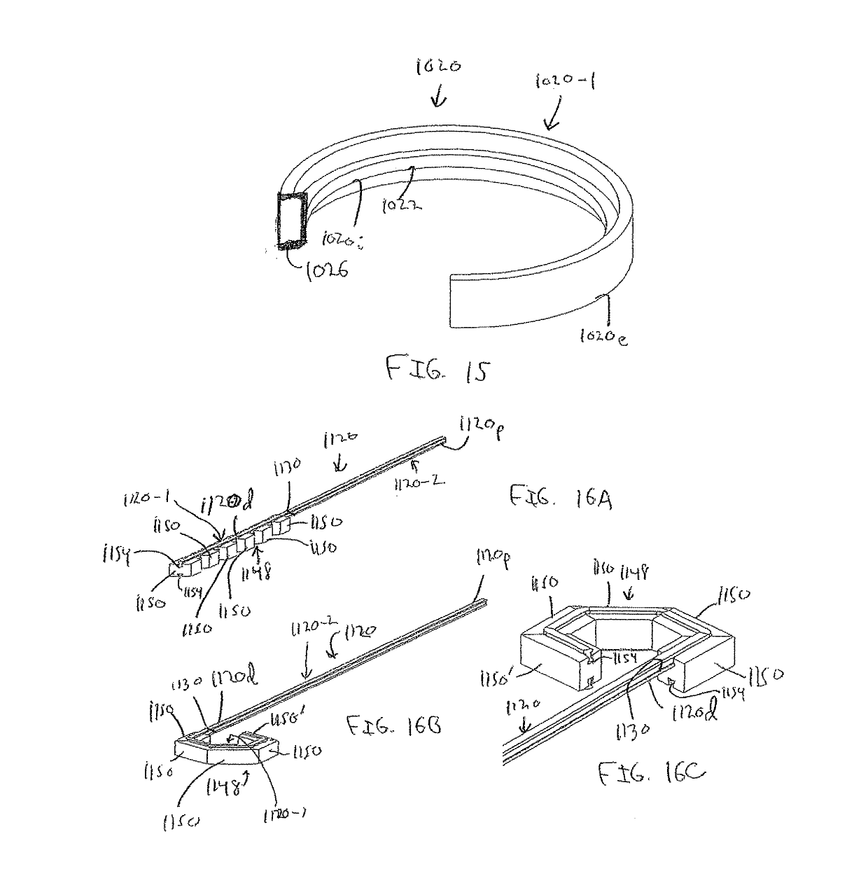

Segments 950, shown in FIGS. 14A and 14B, which are, for example, similar to segments 50 detailed above, but with a straight or linear “T” shaped slot 952 (similar to slot 852 of segment 850 detailed above), are deployed by sliding and advancing distally on the rod 920 as detailed for the rod 20 above (and shown in FIGS. 13A-13D). When the segments 950 are all in proper position on the rod 920, covering the curved section 920-1 (similar to the positioning of the segments 50 shown in FIG. 8D), the straight section 920-2 may be broken away at the weakened portion 930, by force exerted by the user. Additionally, the straight section 920-2 is detachable from the curved section 920-1 by arrangements such as those detailed for rod 20 above.

FIG. 14C shows an alternative segment 950′, similar to segments 950, except where indicated. The segment 950′ includes a slot 952, centrally positioned in the segment 950′, but can be positioned non-centrally, such as that for segment 50′. The segment 950′ includes interfaces (contact surfaces) 966, 967 for stabilizing and limiting motion between segments 950′ when positioned on the rod 920. The interfaces 966, 967 include a protrusion or bulge 966 and an indentation or depression 967, arranged oppositely on opposite sides 970, 971 of the segment 950′. This arrangement allows for locking between segments 950′, as the indentations 967 receive the protrusions 966 when the segments 950′ are brought into contact with each other, such as on rod 920, as shown in FIGS. 13A-13C.

FIG. 15 shows an alternative rod 1020, for example, the curved section 1020-1 (terminating in a distal stop surface 1026) of the rod 1020. The rod 1020 typically also includes a straight section, like that of the rods 20, 920, but also in the same configuration as the curved section 1020-1.

The rod 1020 is, for example, in the form of a rail 1022, such as a “T” shaped rail with the “T” extending inward, and running along the internal surface 1020i of the rod 1020. The rod 1020 is of similar construction to the rods 220, 920, with similar components in the “1000s” and operation of the rod 1020 is in accordance with that described for the rod 920 with the segments 50, as detailed above.

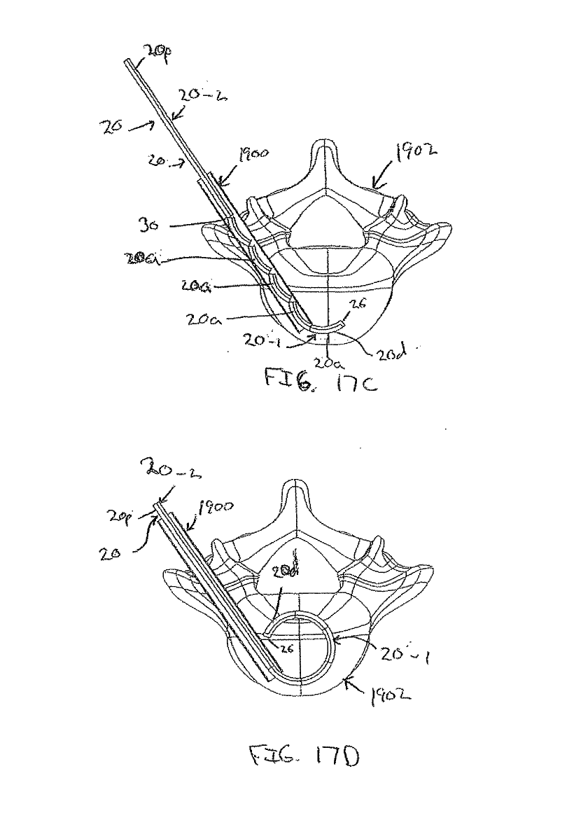

FIGS. 16A-16C show an alternative embodiment including a rod 1120 in the shape of an I-beam, similar to that of rod 820 detailed above, except that the rod 1120 is, for example, integral and in two straight sections 1120-1 (distal), and 1120-2 (proximal), separated by a weakened portion 1130, in accordance with the weakened portion 30 of the rod 20 detailed above. The rod 1120 differs from the rod 820 in that the section 1120-1 corresponding to the curved section 820-1 of rod 820 does not curve, but rather bends angularly (with the rod 1120 linear between the bends), as the segments 1150 bend angularly with respect to each other, as shown in FIGS. 16B and 16C.

The rod 1120 fits into slots 1154 in a member 1148, for example, an integral member, having hinged segments 1150. The hinged segments 1150 of the member 1148 allow for the member 1148 to bend upon deployment into the body (FIGS. 16B, 16C), from an initially straight orientation (FIG. 16A). The bending may be caused by a string or other tensioning element (not shown), which is, for example, attached to the distal-most segment 1150. With the bending complete, the proximal section 1120-2 is broken away from the distal section 1120-1 at the weakened portion 1130.

FIGS. 17A-17G illustrate an exemplary deployment of the apparatus 1910 (FIG. 17G) of the present invention in the body. The apparatus includes the rod 20 detailed above and segments 50 as detailed above. In FIG. 17A a conduit 1900 is inserted into the body, for example, through the back (the patient lies on his belly) in order to access the surgical site, the area between vertebrae 1902. Access by the conduit 1900 is in accordance with standard surgical methods.

A rod, for example, rod 20, shown in FIGS. 1A, 1B and 2 and described above, is inserted into the conduit 1900, as shown in FIG. 17B. The rod 20 is then advanced out of the conduit 1900, with the advancement causing links 20a to deflect, as shown in FIG. 17C, until all of its links 20a, exit the conduit 1900 and have deflected, into a curved, rounded or coiled configuration, as shown in FIG. 17D. Advancement of the rod 20 in the conduit 1900 also ceases at this time, as the rod 20 is in its desired coiled, rounded or curved position. The conduit 1900 is then removed from the body by conventional surgical procedures.

Segments 50 are then slid over the rod 20, such that the segments 50 are advanced distally on the rod 20 (toward the distal end 20d), as shown in FIG. 17E. Segments 50 are added and advanced distally along the rod 20, by sliding along the rail 22. This process of adding segments 50 and advancing them distally, by sliding the segments 50 along the rod 20 continues until a desired configuration of segments 50 is reached, for example, the segments 50 cover the curved section 20-1 of the rod 20, as shown in FIG. 17F. The straight section 20-2 of the rod 20 is exposed, and now ready to be separated from the curved section 20-1, for example, by being broken away from the curved section 20-1.

In FIG. 17G, the straight section 20-2 has been successfully separated from the curved section 20-1, and has been removed from the body (by conventional surgical techniques), leaving the curved section 20-1 with segments 50 deployed in the body, at the desired site, for example, the vertebrae 1902. The weakened portion 30 is such that upon the breaking away, one or more edges of the curved section 20-1 extend outward, serving as a proximal stop for the segments 50. The curved section 20-1 with the segments 50 thereon defines an implant (apparatus) 1910 on which further procedures, such as inserting bone fill or other fusion promoting materials can be inserted, is performed.

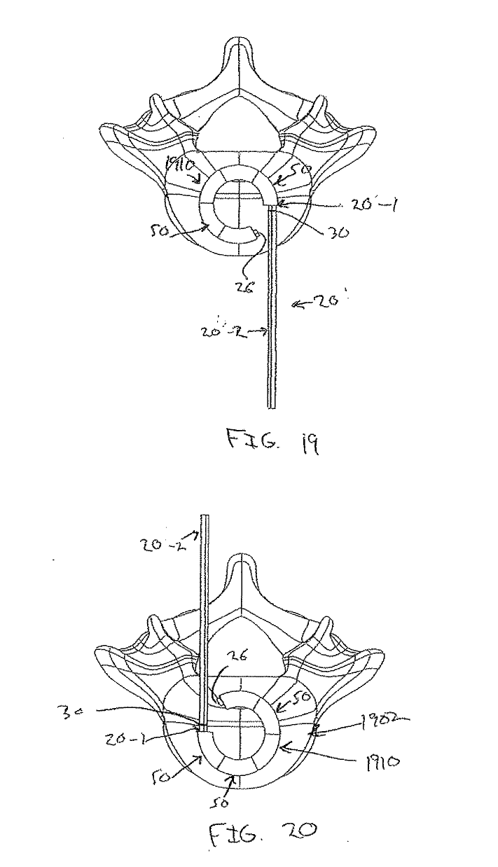

FIG. 18 shows the procedure of FIGS. 17A-17G, except that deployment of the rod 20 and segments 50 is from access provided at the side of the body. For this side access, the patient lies on his belly or side.

FIG. 19 shows the procedure of FIGS. 17A-17G, except that deployment of the rod 20 and segments 50 is from access provided at the front of the body. For this front access, the patient lies on his back.

FIG. 20 shows the procedure of FIGS. 17A-17G, except that deployment of the rod 20 and segments 50 is from access provided at another point along the back of the body. For this access, the patient lies on his belly.

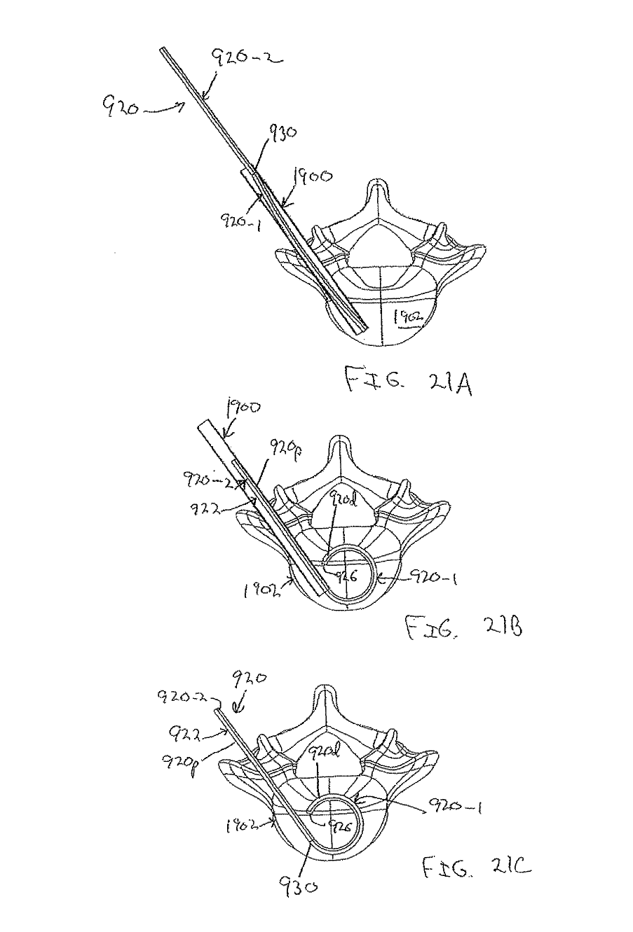

FIGS. 21A-21C show a deployment of the rod 920, as detailed above, and shown in FIGS. 13A-13D, and segments 950, as shown in FIGS. 13A-13D and 14A and 14B. The rod 920 is in an uncoiled state in the conduit 1900, as shown in FIG. 21A. The rod 920 is advanced distally, out of the conduit 1900, where it deflects to its curved or coiled shape, as shown in FIG. 21B. With the conduit 1900 removed, as shown in FIG. 21C, segments 950 are deployed over the rod 920 by being advanced distally by sliding. Deployment continues in accordance with FIGS. 17A-17G, as detailed above.

Additional procedures which can be performed using the apparatus detailed above, for example, include interbody fusion, vertebral compression fracture stabilization, such as vertebroplasty and kyphoplasty, disc replacement, and the like. While the apparatus and methods of the invention are shown for a human, the disclosed apparatus and methods are also suitable for use with other animals, including mammals.

While preferred embodiments of the present invention have been described, so as to enable one of skill in the art to practice the present invention, the preceding description is intended to be exemplary only. It should not be used to limit the scope of the invention, which should be determined by reference to the following claims.

1. A surgical implant for use in the body comprising:
a rod including a first section configured for deflecting into a predetermined non-linear shape when deployed in the body, from a non-deflected substantially linear shape, and a second section, the rod configured to be separated between the first section and the second section; and,
at least one segment configured for movement along the first and second sections of the rod, and configured to conform with the non-linear shape of the first section of the rod for positioning thereon, when the first section of the rod is deflected upon being deployed in the body.

2. The surgical implant of claim 1, wherein the second section of the rod is linear.

3. The surgical implant of claim 2, wherein an engagement portion extends along the rod, and the at least one segment is correspondingly configured to the cross sectional shape of the engagement portion for slideably interlocking with the engagement portion along the rod.

4. The surgical implant of claim 2, wherein the first section of the rod includes a plurality of flexibly connected links.

5. The surgical implant of claim 4, wherein the links are of the same length.

6. The surgical implant of claim 5, wherein the at least one segment is of a length greater than the combined length of the links.

7. The surgical implant of claim 6, wherein the at least one segment includes a plurality of segments, each of the segments of a length such that each of the separations between the links are overlapped by a segment of the plurality of segments.

8. The surgical implant of claim 3, wherein the engagement portion is selected from the group consisting of a T-shaped rail and a track of a U-shaped cross section.

9. The surgical implant of claim 8, wherein the at least one segment includes a slot of a correspondingly configured T-shape to the T-shape of the engagement portion of the rod.

10. The surgical implant of claim 9, wherein the slot extends in an arc through the at least one segment.

11. The surgical implant of claim 9, wherein the slot extends linearly through the segment.

12. The surgical implant of claim 8, wherein the engagement portion of the rod is on the exterior surface of the rod.

13. The surgical implant of claim 8, wherein the engagement portion of the rod is on the interior surface of the rod.

14. The surgical implant of claim 2, wherein the rod includes an 1-beam shaped member, and the at least one segment includes a plurality of segments, each of the segments including slots at oppositely disposed ends to engage a portion of the 1-beam shaped member.

15. The surgical implant of claim 14, wherein the slots extend linearly through the segment

16. The surgical implant of claim 14, wherein the slots extend in an arc through the at least one segment.

17. The surgical implant of claim 1, wherein the first section of the rod with the at least one segment placed along the rod define the portion of the surgical implant that remains in the body.

18. The surgical implant of claim 1, wherein the first section of the rod includes a shape-memory material which is configured to deflect into the predetermined non-linear shape when deployed in the body.

19. The surgical implant of claim 18, wherein the non-linear shape is selected from the group consisting of curved and rounded.

20. The surgical implant of claim 7, wherein each of the segments of the plurality of segments includes correspondingly configured engaging structures at oppositely disposed ends of the segment.

21. The surgical implant of claim 1, wherein the second section of the rod is rigid.

22. A method for deploying a surgical implant to a surgical site in the body comprising:
accessing the surgical site with a tube;
moving a rod through the tube, whereby the leading section of the rod deflects into a predetermined non-linear shape upon exiting the tube at the surgical site;
removing the tube from enveloping the rod;
moving at least one segment along the rod toward the edge of the leading end, to define the surgical implant;
separating a section of the rod from the leading section of the rod; and,
removing the separated section of the rod from the body.

23. The method of claim 22, wherein the at least one segment includes a plurality of segments, and moving the at least one segment includes moving each segment of the plurality of segments until the leading segment is at the edge of the leading end with the segments in abutment with each other.

24. The method of claim 23, additionally comprising: each segment of the plurality of segments engaging a successive segment of the plurality of segments in an interlocking manner at the oppositely disposed ends of the segments.
